(19) United States
(12) Patent Application Publication
(10) Pub. No.: US 2025/0186066 A1
(43) Pub. Date: Jun. 12, 2025

(54) ROTATING JET FOR THROMBECTOMY CATHETER

(71) Applicant: WALK VASCULAR, LLC, Irvine, CA (US)

(72) Inventors: Michael Tizio, Westford, MA (US); Matthew J. Gillick, Murrieta, CA (US)

(21) Appl. No.: 18/965,805

(22) Filed: Dec. 2, 2024

Related U.S. Application Data
(60) Provisional application No. 63/607,734, filed on Dec. 8, 2023.

(51) Int. Cl. A61B 17/22; A61B 17/00

(52) U.S. Cl. CPC A61B 17/22; A61B 17/00234; A61B 2017/00238; A61B 2017/00292; A61B 2017/00539; A61B 2017/22079; A61B 2217/005; A61B 2217/007

(57) ABSTRACT

Systems and methods including a jet rotator (e.g., a saline jet rotator) to aspirate a thrombus or thrombotic material. The jet rotator rotates when the pressurized fluid (e.g., saline) is allowed to flow through the supply lumen. The jet rotator includes a proximal plenum that is configured to allow injection of the pressurized fluid into the jet rotator when the pressurized fluid flows through the supply lumen. The jet rotator includes a distal plenum and one or more pressurized fluid channels. The channels allow the pressurized fluid to flow from the proximal plenum to the distal plenum. The jet rotator includes a jet orifice that is configured to allow the pressurized fluid to flow from the distal plenum into the aspiration lumen. Either the jet orifice, the channels, or other features of the jet rotator provide for rotation of the jet rotator relative to the aspiration lumen during use.

### CROSS-REFERENCE TO RELATED APPLICATIONS

This application claims the benefit of and priority to U.S. Provisional Patent Application Ser. No. 63/607,734 filed Dec. 8, 2023, and entitled “ROTATING JET FOR THROMBECTOMY CATHETER,” which application is expressly incorporated herein by reference in its entirety.

### BACKGROUND

### 1. The Field of the Invention

The present disclosure relates to removal of clots in blood vessels, which employ a combination of suction through a catheter and an internal high-pressure saline jet within the catheter to macerate clot tissue during aspiration. In more detail, embodiments of the invention relate to devices that include a jet rotator to better break up thrombotic material.

### 2. The Relevant Technology

Several devices and systems already exist to aid in the removal of thrombotic material. These include simple aspiration tube type devices using vacuum syringes to extract thrombus into the syringe, simple flush-and-aspirate devices, more complex devices with rotating components that pull in, macerate and transport thrombotic material away from the distal tip using a mechanical auger, and systems that use very high pressure to macerate the thrombus and create a venturi effect to flush the macerated material away.

All of the devices described above have limitations as a result of individual design characteristics. For example, simple aspiration catheters offer ease of use and rapid deployment but may become blocked or otherwise inoperable when faced with older, more organized thrombotic material. Such devices must be removed and cleared outside the body and then re-inserted into the vasculature, which lengthens the time needed for the procedure and increases the opportunity to kink the catheter shaft. Such kinks may reduce performance by decreasing the cross-sectional area of the catheter or may render the device inoperable.

Mechanical rotary devices use an auger to grab and carry the thrombus away from the target area. Some create transport force via vacuum bottles while others create differential pressure at the distal tip of the device with the auger acting as a low-pressure pump. These devices typically work slowly and offer the physician no feedback as to when the device should be advanced further into the lesion.

Flushing type devices include manual flush type devices in which the physician manipulates a hand-driven pump to provide flowing saline at the tip of the device to break up and aspirate the thrombus material, which may introduce performance variations based on the ability of the physician to consistently pump the device over the duration of the procedure.

Another system uses a stationary high-pressure saline jet, which is directed radially towards the center of the catheter. The jet is small in cross-section relative to the catheter, e.g., approximately 0.002″×0.006″, while the catheter is significantly larger (e.g., approximately 0.080″ diameter). With such a configuration, it is possible for a blood clot to clog the catheter as a result of incomplete maceration by the saline jet due to the size difference.

Accordingly, there is an ongoing need for improved systems to remove thrombotic material.

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

An embodiment of the present disclosure relates to a system for aspirating thrombus, including: an elongate shaft configured for placement within a blood vessel of a subject; a supply lumen and an aspiration lumen each extending along the shaft; a pressurized fluid source in fluid communication with the supply lumen to provide pressurized fluid to the supply lumen; and a jet rotator. The jet rotator rotates when the pressurized fluid is allowed to flow through the supply lumen. The jet rotator includes a proximal plenum and a distal plenum. The proximal plenum is configured to allow injection of the pressurized fluid into the jet rotator when the pressurized fluid is caused or allowed to flow through the supply lumen. The jet rotator also includes one or more pressurized fluid channels, wherein the channels allow the pressurized fluid to flow from the proximal plenum to the distal plenum. A jet orifice is also provided, where the jet orifice is configured to allow the pressurized fluid to flow from the distal plenum into the aspiration lumen.

Another embodiment of the present disclosure relates to a method for aspirating thrombus, the method including providing a system for aspirating a thrombus. The system includes an elongate shaft configured for placement within a blood vessel of a subject and a supply lumen and an aspiration lumen each extending along the shaft. A pressurized fluid source is provided in fluid communication with the supply lumen to provide pressurized fluid to the supply lumen. A jet rotator is also provided, where the jet rotator rotates when the pressurized fluid is allowed to flow through the supply lumen. The jet rotator includes a proximal plenum and a distal plenum. The proximal plenum is configured to allow injection of the pressurized fluid into the jet rotator when the pressurized fluid is caused or allowed to flow through the supply lumen. One or more pressurized fluid channels are also provided, wherein the channels allow the pressurized fluid to flow from the proximal plenum to the distal plenum. Finally, a jet orifice is also provided, wherein the jet orifice is configured to allow the pressurized fluid to flow from the distal plenum into the aspiration lumen. The method further includes flowing the pressurized fluid to the supply lumen from the pressurized fluid source, wherein the pressurized fluid causes the jet rotator to rotate, macerating a thrombus located in the elongate lumen when the pressurized fluid contacts the thrombus, and aspirating the macerated thrombus.

Another embodiment of the present disclosure relates to a jet rotator, wherein the jet rotator rotates when a pressurized fluid is allowed to flow through an associated supply lumen. The jet rotator includes a proximal plenum, where the proximal plenum is configured to allow injection of a pressurized fluid into the jet rotator when the pressurized fluid is caused or allowed to flow through a supply lumen. A distal plenum, and one or more pressurized fluid channels are also provided, where the channels allow the pressurized fluid to flow from the proximal plenum to the distal plenum. The jet rotator also includes a jet orifice, wherein the jet orifice is configured to allow the pressurized fluid to flow from the distal plenum into an associated aspiration lumen.

In any of the described embodiments, the jet rotator may be located radially towards a center of the elongate shaft.

In any of the described embodiments, the jet orifice may have a cross-sectional dimension from about 0.002 inches to about 0.006 inches.

In any of the described embodiments, the elongate shaft may have an inner diameter of about 0.080 inches.

In any of the described embodiments, the supply lumen may provide the pressurized fluid at a pressure within a range of about 3 MPa to about 6 MPa.

In any of the described embodiments, the one or more pressurized fluid channels may have a helical shape.

In any of the described embodiments, the pressurized fluid flowing through the channels may apply a rotational force on the jet rotator causing the jet rotator to rotate.

In any of the described embodiments, the jet orifice may be about 0.002 inches by about 0.006 inches.

In any of the described embodiments, the jet rotator may be located within the elongate shaft.

In any of the described embodiments, a small gap may exist between the jet rotator and the elongate shaft, wherein some pressurized fluid may leak between the jet rotator and the elongate shaft to form a hydrodynamic bearing therebetween. By way of example, the gap may be within a range from about 0.0005 inches to about 0.005 inches.

In any of the described embodiments, the hydrodynamic bearing may be configured to allow free rotation of the jet rotator.

In any of the described embodiments, the jet orifice may direct a pressurized jet of the pressurized fluid into the aspiration lumen, e.g., substantially centered on a centerline of the aspiration lumen.

In any of the described embodiments, the jet orifice may be offset relative to a centerline of the aspiration lumen.

In any of the described embodiments, the pressurized fluid flowing through the jet orifice may apply a rotational force to the jet rotator causing the jet rotator to rotate.

Any of such features described in any given embodiment may be used in combination with any feature described in any other embodiment.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

A description of various aspects and features of the invention will be rendered by reference to various representative embodiments thereof illustrated in the appended drawings. It is appreciated that these drawings depict only typical embodiments of the invention and are therefore not to be considered limiting of its scope.

FIG. 1 is a plan view of exemplary disposable components of a system for aspirating thrombus according to an implementation of the present disclosure.

FIG. 2 is a cross-sectional view of an exemplary distal end of the aspiration catheter of the system for aspirating thrombus of FIG. 1 according to an implementation of the present disclosure.

FIG. 3 is a detail view of an exemplary y-connector of the aspiration catheter of the system for aspirating thrombus of FIG. 1 according to an implementation of the present disclosure.

FIG. 4 is a perspective view of an exemplary system for aspirating thrombus using system components such as those of FIG. 1 according to an implementation of the present disclosure.

FIG. 5 is an exploded view of a portion of the system of FIG. 4 according to an implementation of the present disclosure.

FIG. 6 is a perspective view of an exemplary aspiration catheter according to an implementation of the present disclosure.

FIG. 7 is a cross-sectional view of an exemplary aspiration catheter according to an implementation of the present disclosure.

FIG. 8 schematically illustrates the exemplary aspiration catheter within a body lumen aspirating thrombus according to an implementation of the present disclosure.

FIG. 9 illustrates an exemplary jet rotator including a plurality of helical channels for rotation, according to an implementation of the present disclosure.

FIG. 10 illustrates an exemplary jet rotator including an offset jet orifice, so as to provide for rotation of the jet rotator, according to an implementation of the present disclosure.

FIGS. 11-12 illustrate a cross-sectional view and a plan view, respectively, of the jet rotator of FIG. 10, showing the offset jet orifice, and how it directs the pressurized fluid jet so as to be offset, relative to the centerline of the aspiration lumen.

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

One or more embodiments of the present disclosure may generally relate to a jet rotator that rotates when a pressurized fluid is allowed to flow through a supply lumen associated with the jet rotator. The pressurized fluid enters the jet rotator from the supply lumen, into a proximal plenum of the jet rotator, and eventually exits the jet rotator, entering into the aspiration lumen, so as to break apart thrombotic material or a thrombus located in the aspiration lumen. The jet of pressurized fluid rotates relative to the aspiration lumen as a result of the jet rotator, and breaks apart the thrombotic material or thrombus more efficiently by contacting the thrombotic material or thrombus from multiple directions with the pressurized fluid, during aspiration. The jet rotator rotates as a result of the pressurized fluid passing through the jet rotator as supplied from the supply lumen in a manner that causes a rotational force to be applied to the rotator. The described configurations are simple, and do not necessarily require any additional mechanical parts or human intervention to cause rotation.

While the present disclosure will describe various particular implementations of a jet rotator that breaks apart thrombotic material or thrombus located in an aspirational lumen, it should be understood that the devices, systems, and method described herein may be applicable in other environments, and to other uses. Additionally, elements described in relation to any embodiment depicted and/or described herein may be combinable with elements described in relation to any other embodiment depicted and/or described herein.

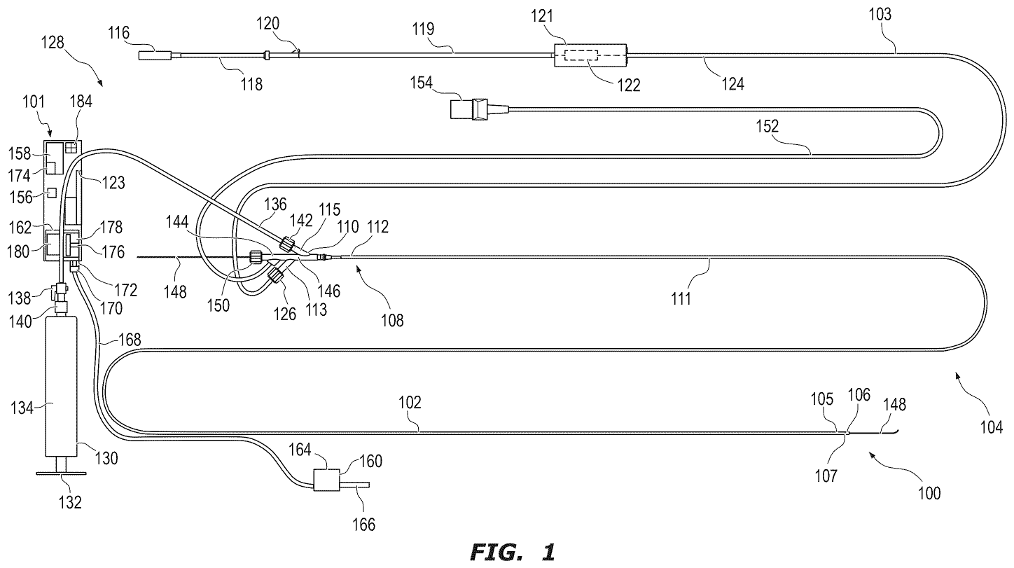

A system 100 for aspirating thrombus is illustrated in FIGS. 1-5, The system 100 for aspirating thrombus includes a pump 101, an aspiration catheter 102, and a tubing set 103. The aspiration catheter 102 and the tubing set 103 represent disposable components 104, and the pump 101, and the pump's associated pump base, is a reusable component. It is not necessary to sterilize the pump 101 as it may be kept in a non-sterile field or area during use. The aspiration catheter 102 and the tubing set 103 may each be supplied sterile, after sterilization by ethylene oxide gas, electron beam, gamma, or other sterilization methods. The aspiration catheter 102 may be packaged and supplied separately from the tubing set 103, or the aspiration catheter 102 and the tubing set 103 may be packaged together and supplied together. Alternatively, the aspiration catheter 102 and tubing set 103 may be packaged separately, but supplied together (i.e., bundled).

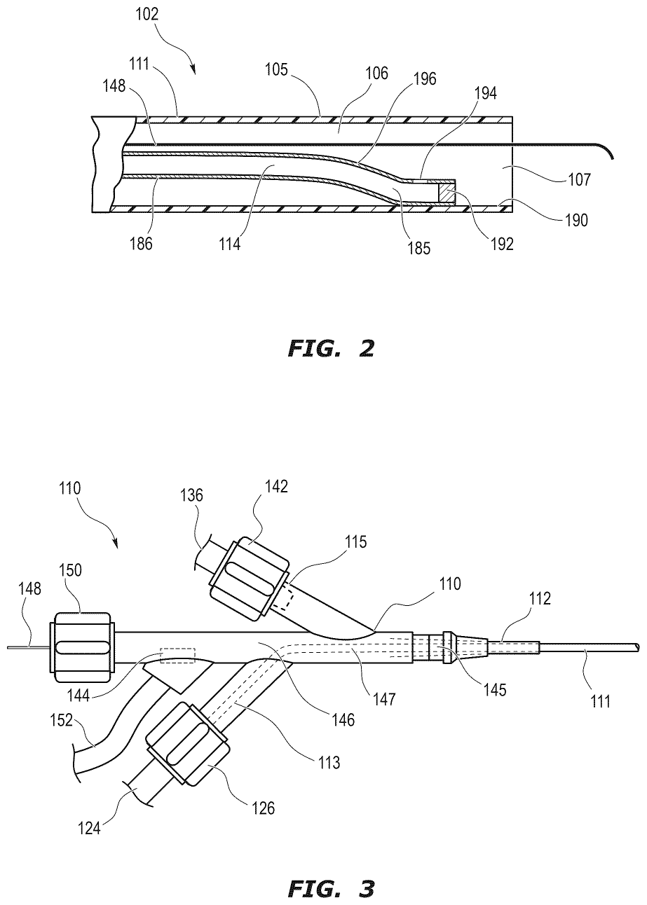

The aspiration catheter 102 has a distal end 105 and includes an over-the-wire guidewire lumen/aspiration lumen 106 extending between an open distal end 107, and a proximal end 108 comprising or connected to a y-connector 110. The catheter shaft 111 of the aspiration catheter 102 is connected to the y-connector 110 via a protective strain relief 112. In other embodiments, the catheter shaft 111 may be attached to the y-connector 110 with a luer fitting. The y-connector 110 includes a first female luer 113 which communicates with a catheter supply lumen 114 (FIG. 2), and a second female luer 115 which communicates with the guidewire lumen/aspiration lumen 106.

A spike 116 for coupling to a fluid source (not shown) (e.g., saline bag, saline bottle) allows fluid to enter through an extension tubing 118 and flow into a supply tube 119. An optional injection port 120 allows injection of materials or removal of air. A cassette 121 having a moveable piston 122 is used in conjunction with a mechanical actuator 123 of the pump 101. Fluid is pumped into an injection tube 124 from action of the cassette 121 as applied by the actuator 123 of the pump 101. A male luer 126, hydraulically communicating with the catheter supply lumen 114, via the injection tube 124, is configured to attach to the female luer 113 of the y-connector 110.

Accessories 128 are illustrated that are intended for applying a vacuum source, such as a syringe 130 having a plunger 132 and a barrel 134, to the aspiration lumen 106 of the aspiration catheter 102. The syringe 130 is attached to a vacuum line 136 via the luer 140 of the syringe 130. A stopcock 138 may be used on the luer 140 to maintain the vacuum, or alternatively, the plunger 132 may be a locking variety of plunger that is configured to be locked in the retracted (vacuum) position. A male luer 142 at the end of the vacuum line 136 may be detachably secured to the female luer 115 of the y-connector 110 of the aspiration catheter 102. As shown in more detail in FIG. 3, a pressure sensor (e.g., transducer) 144 is secured inside an internal cavity 146 of the y-connector 110 proximal to the female luer 113 and the female luer 115. A valve 150, for example a Touhy-Borst, at the proximal end of the y-connector 110 allows hemostasis of the guidewire lumen/aspiration lumen 106 around a guidewire 148. In other embodiments, the valve 150 may comprise a longitudinally spring-loaded seal. The guidewire 148 may be inserted entirely through the guidewire lumen/aspiration lumen 106. Signals output from the pressure sensor 144 are carried through a cable 152 to a connector. The connector is plugged into a socket 156 of the pump 101. Pressure related signals may be processed by a circuit board 158 of the pump 101. The pressure sensor or transducer 144 may be powered from the pump 101, via the cable 152. The accessories 128 may also be supplied sterile to the user.

A foot pedal 160 is configured to operate a pinch valve 162 for occluding or opening the vacuum line 136. The foot pedal 160 comprises a base 164 and a pedal 166 and is configured to be placed in a non-sterile area, such as on the floor, under the procedure table/bed. The user steps on the pedal 166 causing a signal to be sent along a cable 168 which is connected via a plug 170 to an input jack 172 in the pump 101. The vacuum line 136 extends through a portion of the pump 101. The circuit board 158 of the pump may include a controller 174 configured to receive one or more signals indicating on or off from the foot pedal 160. The controller 174 of the circuit board 158 may be configured to cause an actuator 176 carried by the pump 101 to move longitudinally to compress and occlude the vacuum line 136 between an actuator head 178 attached to the actuator 176 and an anvil 180, also carried by the pump 101. By stepping on the pedal 166, the user is able to thus occlude the vacuum line 136, stopping the application of a negative pressure. In some embodiments, as the pedal 166 of the foot pedal 160 is depressed, the controller may be configured to open the pinch valve 162.

The pressure sensor or transducer 144 thus senses a negative pressure and sends a signal, causing the controller to start the motor of the pump 101. As the effect via the electronics is substantially immediate, the motor starts pumping almost immediately after the pedal 166 is depressed. As the pedal 166 of the foot pedal 160 is released, the controller 174 then causes the pinch valve 162 to close. The pressure sensor or transducer 144 thus senses that no negative pressure is present and the controller 174 causes the motor of the pump 101 to shut off. Again, the effect via the electronics is substantially immediate, and thus the motor stops pumping almost immediately after the pedal 166 is depressed. During sterile procedures, the main interventionalist is usually “scrubbed” such that the hands only touch items in the sterile field. However, the feet/shoes/shoe covers are not in the sterile field. Thus again, a single user may operate a switch (via the pedal 166) while also manipulating the aspiration catheter 102 and guidewire 148. However, this time, it is the sterile field hands and non-sterile field feet that are used. Alternatively, the foot pedal 160 may comprise two pedals, one for occlude and one for open. In an alternative foot pedal embodiment, the pedal 166 may operate a pneumatic line to cause a pressure activated valve or a cuff to occlude and open the vacuum line 136, for example, by forcing the actuator head 178 to move. In another alternative embodiment, the pedal 166 may turn, slide, or otherwise move a mechanical element, such as a flexible pull cable or push rod that is coupled to the actuator 176, to move the actuator head 178. The cable 168 may be supplied sterile and connected to the base 164 prior to a procedure. The occlusion and opening of the vacuum line 136 thus acts as an on and off switch for the pump 101 (via the pressure sensor 144). The on/off function may thus be performed by a user whose hands can focus on manipulating sterile catheters, guidewires, and accessories, and whose foot can turn the pump on and off in a non-sterile environment. This allows a single user to control the entire operation or the majority of operation of the system 100 for aspirating thrombus. This can be an advantage both in terms of a rapid, synchronized procedure, but is also helpful in laboratories where additional assistants are not available. The actuator 176 and anvil 180 may be controlled to compress the vacuum line 136 with a particular force, and the actuator 176 may be controlled to move at a particular speed, either when compressing or when removing compression. Speed and force control allows appropriate response time, but may also be able to add durability to the vacuum line 136, for example, by not over-compressing. The foot pedal 160 may communicate with the pinch valve 162 via a wired connection through the pump 101 or may communicate with the pinch valve 162 wirelessly. Additionally, or alternatively, the pump may be controlled by buttons 184.

It should be noted that in certain embodiments, the pinch valve 162 and the foot pedal 160 may be incorporated for on/off operation of the pinch valve 162 on the vacuum line 136, without utilizing the pressure sensor 144. In fact, in some embodiments, the pressure sensor 144 may even be absent from the system 100 for aspirating thrombus, the foot pedal 160 being used as a predominant control means.

Turning to FIG. 2, a supply tube 186, which contains the catheter supply lumen 114, freely and coaxially extends within the over-the-wire guidewire lumen/aspiration lumen 106. At least a distal end 185 of the supply tube 186 is secured to an interior wall 190 of the guidewire lumen/aspiration lumen 106 of the catheter shaft 111 by adhesive, epoxy, hot melt, thermal bonding, or other securement modalities. A plug 192 is secured within the catheter supply lumen 114 at the distal end 185 of the supply tube 186. The plug 192 blocks the exit of pressurized fluid, and thus the pressurized fluid is forced to exit through an orifice (e.g., opening) 194 in the wall 196 of the supply tube 186. The free, coaxial relationship between the supply tube 186 and the catheter shaft 111 along their respective lengths, allows for improved flexibility. In some embodiments, in which a stiffer proximal end of the aspiration catheter 102 is desired (e.g., for pushability or even torqueability), the supply tube 186 may be secured to the interior wall 190 of the guidewire lumen/aspiration lumen 106 of the catheter shaft 111 along a proximal portion of the aspiration catheter 102, but not along a distal portion. This may be appropriate if, for example, the proximal portion of the aspiration catheter 102 is not required to track through tortuous vasculature, but the distal portion is required to track through tortuous vasculature. The free, substantially unconnected, coaxial relationship between the supply tube 186 and the catheter shaft 111 along their respective lengths, may also be utilized to optimize flow through the guidewire lumen/aspiration lumen 106, as the supply tube 186 is capable of moving out of the way due to the forces of flow (e.g., of thrombus/saline) over its external surface, such that the remaining inner luminal space of the guidewire lumen/aspiration lumen 106 self-optimizes, moving toward the lowest energy condition (least fluid resistance) or toward the largest cross-sectional space condition (e.g., for accommodating and passing pieces of thrombus).

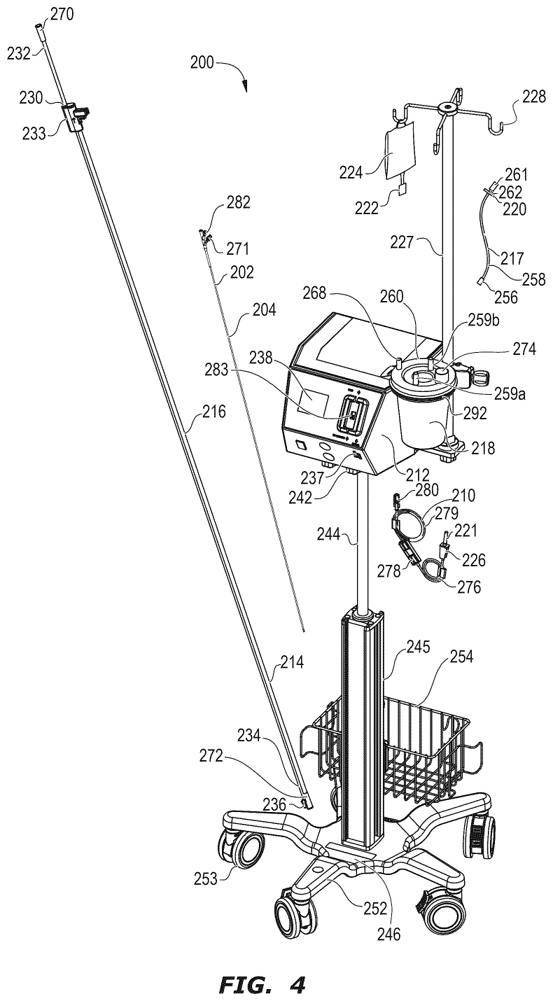

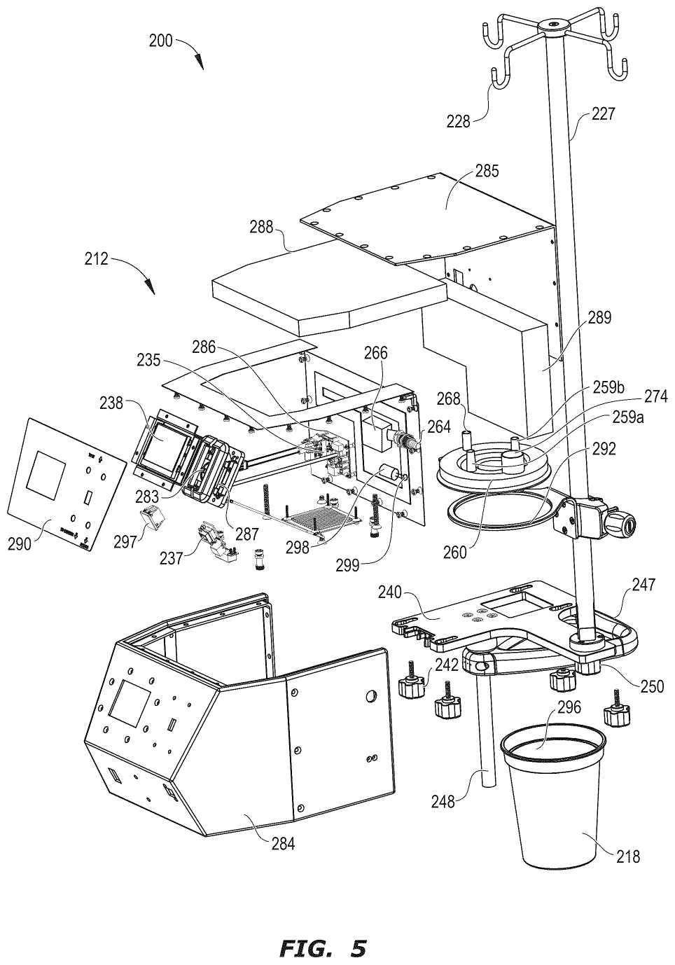

A system 200 for aspirating thrombus is illustrated in FIGS. 4-5. The system 200 for aspirating thrombus is similar to the system 100 and so the disclosure related to the system 100 is also applicable to the description of system 200. An aspiration catheter 202 is similar to the aspiration catheter 102 of FIGS. 1-3 and as such the description related to the aspiration catheter 102 of FIGS. 1-3 is also applicable to the description of the aspiration catheter 202.

The aspiration catheter 202 is configured for aspirating thrombus from peripheral vessels, but may also be configured with a size for treating coronary, cerebral, pulmonary or other arteries, or veins. The aspiration catheter 202 and system 200 may be used in interventional procedures, but may also be used in surgical procedures. The aspiration catheter 202 and system 200 may be used in vascular procedures, or non-vascular procedures (other body lumens, ducts, or cavities). The catheter 202 comprises an elongate shaft 204 configured for placement within a blood vessel of a subject; a catheter supply lumen 114 (FIGS. 2-3) and a guidewire/aspiration lumen 106, each extending along the shaft, the supply lumen 114 having a proximal end 147 and a distal end 185, and the aspiration lumen 106 having a proximal end 145 (FIG. 3) and an open distal end 107 (FIG. 2); and an orifice (e.g., opening) 194 at or near the distal end 185 of the supply lumen 114, the opening configured to allow the injection of pressurized fluid into the aspiration lumen 106 at or near the distal end 107 of the aspiration lumen 106 when the pressurized fluid is pumped through the supply lumen 114 (FIG. 2). In some embodiments, the orifice (e.g., opening) 194 may be located proximal to the distal end 185 of the supply lumen 114. In some embodiments, the distal end 185 of the supply lumen 114 may comprise a plug 192. A pump set 210 (e.g., tubing set) is configured to hydraulically couple the supply lumen 114 to a pump within a saline drive unit (SDU) 212, for injecting pressurized fluid (e.g., saline, heparinized saline) through the supply lumen 114. Suction tubing 214, comprising sterile suction tubing 216 and non-sterile suction tubing 217, is configured to hydraulically couple a vacuum canister 218 to the aspiration lumen 106. A filter 220 may be carried in-line on the suction tubing 214, for example, connected between the sterile suction tubing 216 and the non-sterile suction tubing 217, or on the non-sterile suction tubing 217. The filter 220 is configured to capture large elements such as large pieces of thrombus or emboli.

The pump set 210 includes a saline spike 221 for connection to a port 222 of a saline bag 224, and an inline drip chamber 226 for visually assessing the movement of saline, as well as keeping air out of the fluid being injected. The saline bag 224 may be hung on an IV pole 227 on one or more hooks 228. A pressure sensor 230 such as a vacuum sensor may be used within any lumen of the pump set 210, the suction tubing 214, the supply lumen 114 or aspiration lumen 106 of the catheter 202, or any other component which may see fluid flow. The pressure sensor 230 is shown in FIG. 4 within a lumen at a junction between a first aspiration tube 232 and a control 233. A cable 234 carries signals output from the pressure sensor 230 to a controller 235 in the SDU 212. A connector 236, electrically connected to the cable 234, is configured to be detachably coupled to a mating receptacle 237 (e.g., input jack) in the SDU 212. The SDU 212 also may have a display 238, including an LCD screen or alternative screen or monitor, in order to visually monitor parameters and status of a procedure. In alternative embodiments, the pressure sensor 230 may be replaced by another type of sensor that is configured to characterize fluid flow. In some embodiments, the sensor is a flow sensor, such as a Doppler flow velocity sensor.

The SDU 212 is held on a mount 240 by four locking knobs 242. The mount 240 is secured to a telescoping rod 244 that is adjustable from a cart base 245 via a cart height adjustment knob or other element 246. The mount 240 and a handle 247 are secured to the rod 244 via an inner post 248 that is insertable and securable within an inner cavity in the rod 244. The IV pole 227 secures to the mount 240 via a connector 250. The base 245 may include legs 252 having wheels 253 (e.g., three or more wheels or four or more wheels) and may be movable via the handle 247. The system 200 may also carry a basket 254 for placement of components, products, documentation, or other items.

In use, a user connects a first connector 256 at a first end 258 of the non-sterile suction tubing 217 to a second port 259b on the lid 260 of the canister 218, and connects a second connector 261 at a second end 262 of the non-sterile suction tubing 217 to a vacuum pump input 264 in the SDU 212. A vacuum pump 266 may be carried within the SDU 212 in order to maintain a vacuum/negative pressure within the canister 218. Alternatively, the vacuum inside the canister 218 may be maintained manually, without a vacuum pump, by evacuating the canister 218 via one or more additional ports 268. A user connects a first connector 270 of the sterile suction tubing 216 to an aspiration luer 271 of the aspiration catheter 202 (similar to luer 115), and connects the second connector 272 of the sterile suction tubing 216 to port 274 in the lid 260 of the canister 218. Connector 236 is then coupled to the mating receptacle 237 in the SDU 212 for communication with the control 233 and/or the pressure sensor 230. For instance, the connector 236 can be snapped into mating receptacle 237 in the SDU 212 for communication with elements of the control 233 and/or for communication with the pressure sensor 230, either via cable 234, and/or additional cables or wires. Alternatively, the connector 236 may couple to the mating receptacle 237 by clipping, friction fitting, vacuum fitting, or other means.

After allowing saline to purge through the supply tube 276, cassette 278, and injection tube 279 of the pump set 210, the user connects the luer connector 280 of the pump set 210 to a luer 282 of the aspiration catheter 202 (similar to luer 113). The cassette 278 (similar to cassette 121) is then attached to a saddle 283 in the SDU 212. The saddle 283 is configured to reciprocate a piston to inject the saline from the IV bag 224 at high pressure, after the cassette 278 is snapped in place, keeping the internal contents (e.g., saline) sterile. Systems configured for performing this type of sterile injection of high-pressure saline are described in U.S. Pat. No. 9,883,877, issued Feb. 6, 2018, and entitled, “Systems and Methods for Removal of Blood and Thrombotic Material”, which is incorporated by reference in its entirety for all purposes. The SDU 212 is enclosed within a case 284 and a case lid 285. The controller 235 may reside on a circuit board 286. Noise from a motor 287 controlling the saddle 283 and from the vacuum pump 266 is abated by internal foam sections 288, 289. The saddle 283 may be moved directly by the motor 287, or may be moved with pneumatics, using a cycled pressurization. An interface panel 290 provides one or more switches 297 and the display 238. Alternatively, the cassette 278 may couple to the saddle 283 by clipping, friction fitting, vacuum fitting, or other means.

FIG. 5 illustrates aspects pertaining to the vacuum canister 218, in which aspirant (e.g., thrombus, blood, saline) that is evacuated from the patient through the aspiration lumen 106 is collected. The canister 218 may be held in a canister mount 292 carried by the IV pole 227, or alternatively carried by any other part of the system 200. A lid 260 is configured to cover a portion of the canister 218, such as in a snapping manner, to close an interior 296 of the canister 218. Alternatively, the lid 260 may couple to the canister 218 by screwing, clipping, friction fitting, or other means.

The lid 260 may comprise two or more ports, including the first port 259a and second port 259b for providing negative pressure/vacuum to the aspiration lumen 106. For example, the lid 260 may comprise two ports, three ports, four ports, or more than four ports. Sterile suction tubing 216 may be connected to the lid 260 of the vacuum canister 218 at a first port 259a for transmitting a negative pressure to the sterile suction tubing 216 and to the aspiration lumen 106 of the aspiration catheter 102. Non-sterile suction tubing 217 may be connected to the lid 260 of the vacuum canister 218 at a second port 259b for providing a negative pressure to the vacuum canister 218. A negative pressure may be provided to the non-sterile suction tubing 217 (and to sterile suction tubing 216 and the aspiration lumen 106 connected therewith) by a vacuum source (e.g., a vacuum pump or syringe). The system 200 may also comprise means for sealing the two or more ports of the lid 260 when not in use, such as one or more port caps. A filter may be placed over an entry to the second port 259b so as to prevent aspirant from traveling along the non-sterile suction tubing 217 from the vacuum canister 218 to the vacuum source.

The vacuum canister 218 preferably has a sufficient volumetric capacity for receiving all aspirant collected during the surgical procedure. Receptacles having a volumetric capacity of approximately 100 cubic inches, or receptacles having a diameter of approximately 5.0 inches and a height of approximately 7.0 inches, have been found to provide sufficient volumetric capacity.

Returning to FIG. 5, a solenoid 298 is carried internally in the SDU 212, and is configured to interface with the interior 296 of the canister 218, via the suction tubing 214, or via any additional tubing. The solenoid 298 is configured to vent the negative pressure inside the canister 218, by opening a valve 299 coupled to the solenoid (mechanically or electromagnetically) that opens the interior 296 of the canister 218 to ambient pressure. The venting allows any foaming of blood or fluid, such as any aspirated liquid, within the canister 218 to be reduced. Foaming can occur during a thrombolysis procedure due to cavitation, as air bubbles are formed. The solenoid 298 is then configured to close the valve 299, to allow negative pressure to again be built up within the interior 296 of the canister 218. The controller 235 is configured to automatically energize the solenoid 298, in order to allow for the degassing/defoaming. For example, the controller 235 may send a signal to energize the solenoid 298 based on the measurement of a targeted negative pressure and/or a targeted time of aspiration cycle. In other cases, the controller 235 can send a signal to energize the solenoid 298 every minute, every five minutes, every ten minutes, etc. Additionally, a user can operate the controller 235, and more generally the controller 174, of the system 200 through the interface panel 290 to initiate degassing/defoaming of the interior 296. The venting may also be able to remove air bubbles inside the other lumens of the catheter and tubing sets.

In some embodiments, the controller 235 can output or send a signal to energize the solenoid 298 to open the valve 299, in order to stop any aspiration, while still allowing the SDU 212 to deliver saline, medication, or saline combined with medication (e.g., thrombolytic drugs), so that the fluids can be delivered out of the open distal end 107 (instead of being aspirated through the aspiration lumen 106).

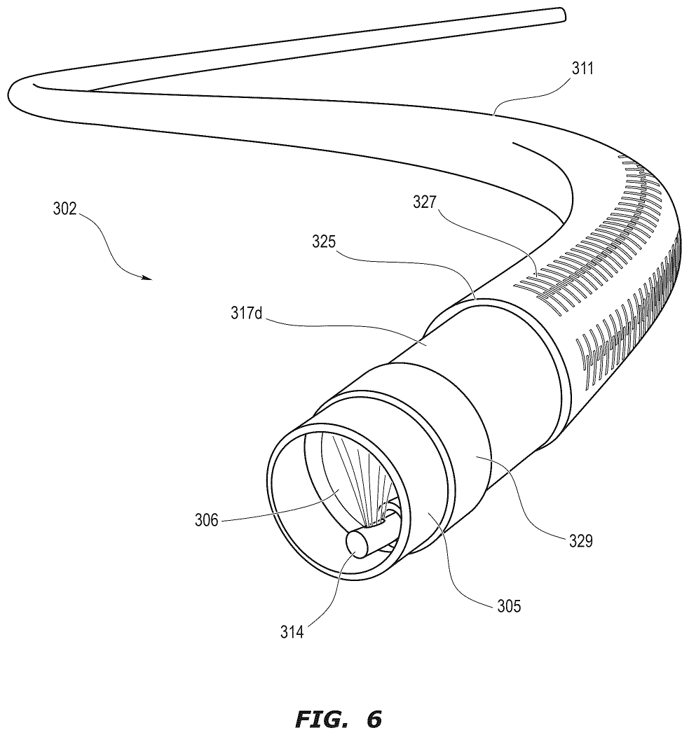

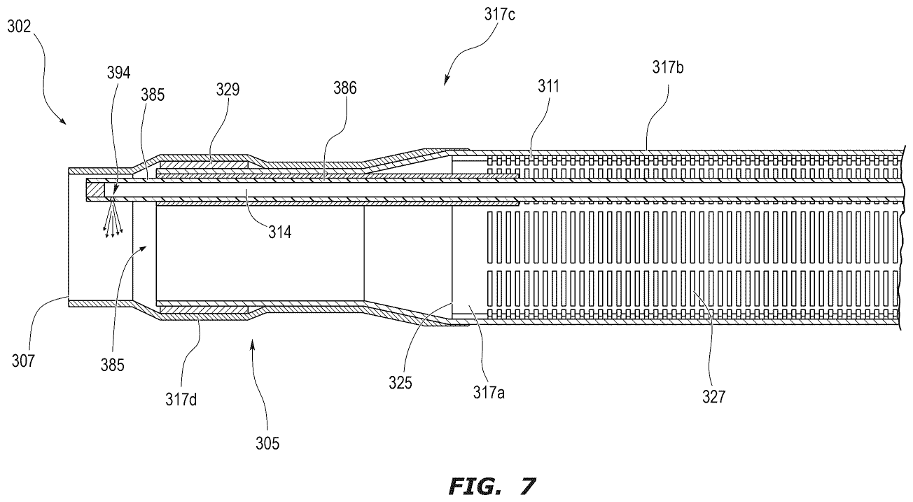

Turning to FIGS. 6-8 illustrated is another configuration of an aspiration catheter 302 that can be used with the systems 100 or 200 for aspirating thrombus. As such, the discussions related to the aspiration catheter 102 and the aspiration catheter 202 are also applicable to the aspiration catheter 302 illustrated in FIGS. 6-8.

As shown in more detail in FIGS. 6-8, aspiration catheter 302 includes an aspiration lumen 306 formed by a shaft 311, such as a hypotube, jacketed by a polymer jacket or a polymer jacket is laminated on the hypotube. For instance, a shaft body 317a is illustrated being jacketed by a jacket 317b. A distal end 305 of the aspiration catheter 302 includes a multilayer structure. A portion 317c of the jacket 317b extends distally of a shaft distal end 325 of the shaft 311 to form part of the distal end 305. An outer layer or outer jacket 317d overlaps the jacket 317b, extends towards and overlaps a shaft distal end 325, and forms the aspiration catheter distal end 305 or the distal most end of the aspiration catheter with the distal opening 307. The outer jacket 317d protects a distal portion 385 of a supply tube 386 containing a supply lumen 314. While reference is made to a multilayer structure, it will be understood that one or more layers can be omitted or combined together. Additionally, one or more of the layers or shaft body can include braided or other members to increase strength and/or flexibility. Alternatively, the shaft and associated layers can be formed by extruding the shaft or using other structure to form the shaft.

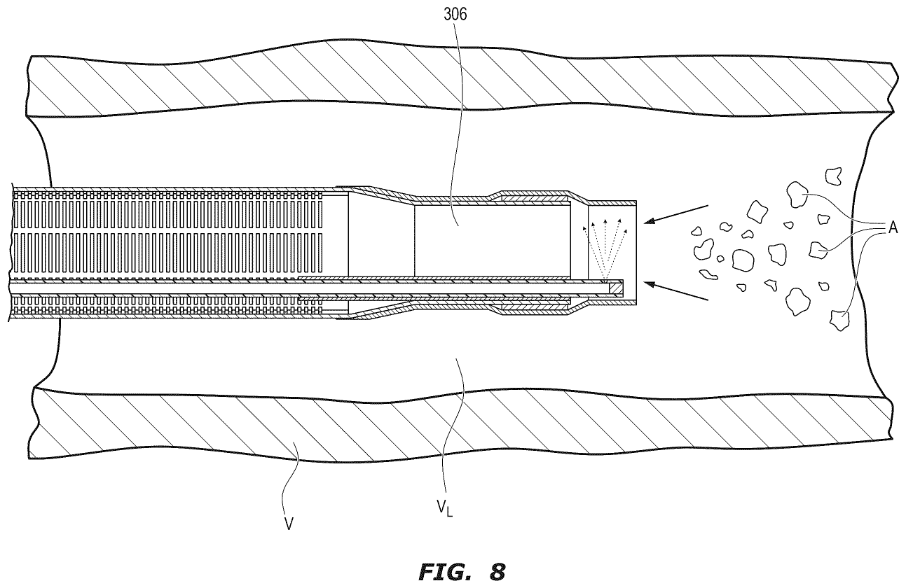

The supply lumen 314 may be configured to provide a high pressure fluid injection, such as saline, within the aspiration lumen 306 for macerating a thrombus as it is aspirated, such as illustrated in FIG. 8 where the aspiration catheter 302 is disposed within a vessel lumen VL of a vessel V and aspirant A is being drawn into the aspiration lumen 306. The saline injection may occur through orifice 394 near the distal end of the supply lumen 314 if the opening of the supply lumen is plugged with a plug similar to plug 192 (FIG. 2). Aspiration catheter 302 may also include a radiopaque (RO) ring 329 at or near the distal end 305 of aspiration catheter 302 for identifying the location of aspiration. The radiopaque ring 329 optionally encircles the aspiration catheter 302. In the illustrated configuration, the RO ring 329 is disposed between the jacket 317b and outer jacket 317d. The RO ring 329 can be formed of or comprise any suitable radiopaque material, such as tantalum, tungsten, platinum/iridium, gold, silver, and combinations or modifications thereof.

The shaft 311 can include one or more openings 327 to increase a flexibility of shaft 311 to aid with advancement of the aspiration catheter 302 through the tortuous anatomy of a patient. While reference is made to a “hypotube,” it will be understood that other tubular structures can be used for the shaft 311. Additionally, the shaft 311 can be formed from polymers, metals, alloys, braided structures, coiled structures, and combinations or modifications thereof. Furthermore, the jacket 317b and outer jacket 317d can be formed of a variety of polymers and copolymers, plastics, PEBAX, HYTREL, rubber, thermoplastic elastomer, other elastomer and combinations or modifications thereof.

In some situations, during aspiration, damage to the vessel might occur because the vessel wall is drawn into the aspiration lumen 306 and comes into contact with the jet of pressurized fluid being injected (e.g., a high pressure saline spray at, for example, 650 psi (4.48 MPa)) from supply lumen 314. By way of example, the supply lumen may provide pressurized fluid (e.g., saline at a pressure from about 3 MPa to about 6 MPa). Therefore, a tissue encroachment prevention assembly can prevent aspiration catheter 302 from damaging the vessel during aspiration. The tissue encroachment prevention assembly can be selectively added to the distal end 305 of the aspiration catheter 302 or the distal end 305 can be modified or changed to accommodate the tissue encroachment prevention assembly.

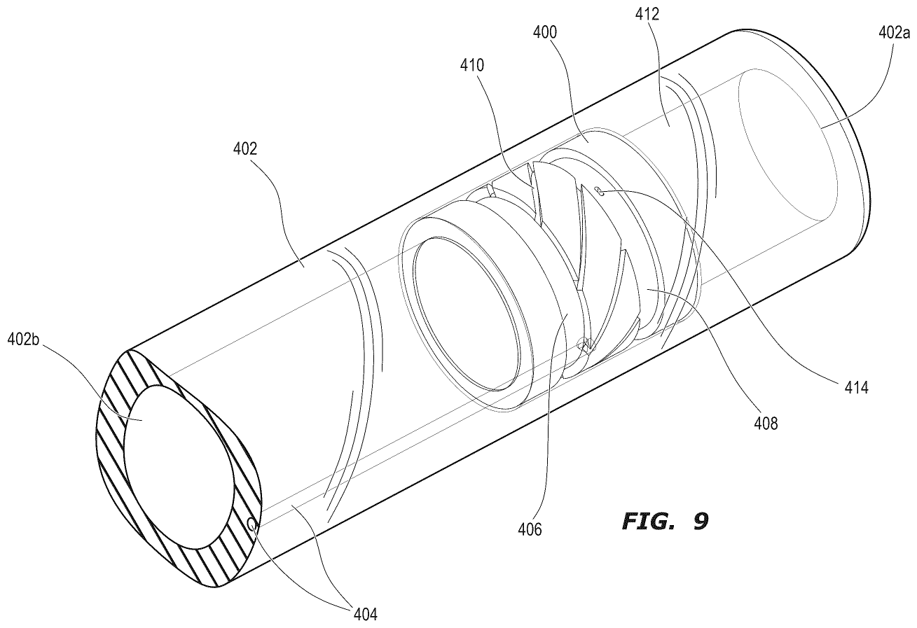

Referring to FIG. 9, illustrated is a sectional view of an exemplary jet rotator 400 that can be implemented into systems 100 or 200 and within the aspiration catheter 302. As shown, the jet rotator 400 is located radially towards the center of the elongate shaft 402 (e.g., aspiration catheter). In some embodiments, the jet rotator 400 is located towards the distal end 402a of the elongate shaft 402 where the high-pressure supply lumen 404 extends along a proximal end 402b of the elongate shaft 402. In yet other embodiments, the jet rotator 400 is located at any point along the elongate shaft 402 and may be statically placed or dynamically (e.g., selectively) moved along the elongate shaft 402.

The supply lumen 404 is in communication with a pressurized fluid source (e.g., saline bag 224 of FIG. 4) which provides the supply lumen 404 with pressurized saline or other fluid. Once the thrombotic material or thrombus is broken apart by the high pressure fluid flowing from the supply lumen 404 through the jet rotator 400, the broken material is vacuumed through the aspiration lumen 412 to a canister (e.g., canister 218 in FIGS. 4-5). The supply lumen 404 and the aspiration lumen 412 may be placed to extend along the elongate shaft 402 which is placed within a blood vessel of a patient or subject, as described in conjunction with the previous Figures.

In some embodiments, the jet rotator 400 has a diameter that fits inside of the aspiration catheter (e.g., about 0.08 inch diameter). In an embodiment, the jet rotator may have a length about 0.05 inch to about 0.25 inch, or from about 0.05 inch to about 0.2 inch, or from about 0.1 inch to about 0.15 inch. In an embodiment, the jet rotor may have an inside diameter of about 0.01 inches, or about 0.05 inches, or about 0.08 inches, or about 0.10 inches and an outside diameter of about 0.1 inches, or about 0.15 inches, or about 0.2 inches. The jet rotator 400 includes a proximal plenum 406 and a distal plenum 408 with one or more fluid channels 410 located between the proximal plenum 406 and the distal plenum 408. The supply lumen 404 is connected to the jet rotator 400 at the proximal plenum 406, so as to deliver the high pressure fluid (e.g., saline) to the proximal plenum 406. When the pressurized fluid is allowed to flow through the supply lumen 404 from the pressurized fluid source, the proximal plenum 406 receives the pressurized fluid into the jet rotator 400. The pressurized fluid flows from the proximal plenum 406 to the distal plenum 408 through the fluid channels 410.

As shown in FIG. 9, the channels 410 can have a helical shape. The helical shape of the illustrated channels 410 imparts a rotational force applied to the jet rotator 400 when the pressurized fluid flows through channels 410, which causes the jet rotator 400 to rotate within the elongate shaft 402 (i.e., the aspiration catheter). Additionally, in some embodiments, a small gap may be provided between the jet rotator 400 and the elongate shaft 402. Such a small gap allows some of the pressurized fluid to leak between the jet rotator 400 and the inner wall of elongate shaft 402 to form a hydrodynamic bearing therebetween. In embodiments, the gap may be about 0.0002 inches, or about 0.00025 inches, or about 0.002 inches, or about 0.0025 inches. The hydrodynamic bearing may allow free rotation of the jet rotator 400 within elongate shaft 402.

As the pressurized fluid flows from the proximal plenum 406 to the distal plenum 408 through the fluid channels 410 causing the jet rotator 400 to rotate, the pressurized fluid passes through jet orifice 414 formed in distal plenum 408, forming a jet of pressurized fluid that sprays into the aspiration lumen 412. Notably, the rotation of the jet rotator ensures that the fluid jet sprayed into the aspiration lumen is not radially stationary relative to the aspiration lumen, but rotates, so as to provide better coverage across the full cross-sectional area of the aspiration lumen. As described with the earlier illustrated embodiments, the pressurized fluid is configured to break apart the thrombotic material or thrombus. By rotating the jet rotator 400, the pressurized fluid can contact the thrombotic material or thrombus in multiple directions causing the material to be broken apart more effectively as compared to a stationary pressurized fluid jet.

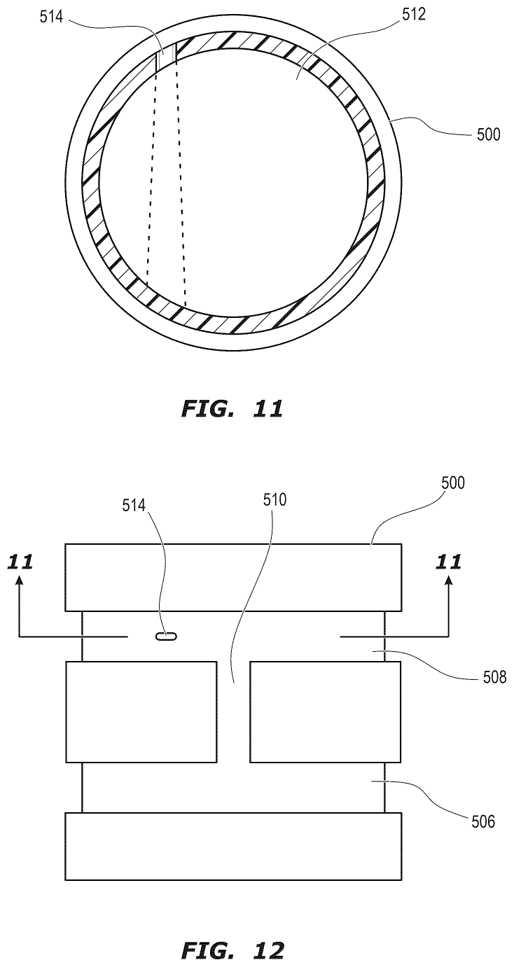

As shown in FIG. 9, the jet orifice 414 can be located on a centerline of the jet rotator 400, and the borehole associated with orifice 414 may be generally normal to the outer surface of plenum 408, so that the pressurized jet is directed radially inward, towards a centerline of the aspiration lumen. On other embodiments, as described in conjunction with FIGS. 11-12, the jet orifice 414 could be offset. In some embodiments, the jet rotator 400 may include one jet orifice 414 or more than one jet orifice 414 (e.g., two, three, four, more than four). In such embodiments, where more than one jet orifice 414 is provided, such jet orifices may be located equally spaced around the perimeter of the jet rotator 400 or they may be unevenly spaced around the perimeter of the jet rotator 400. By way of example, the jet orifice 414 may be about 0.001 inch to about 0.01 inch, about 0.0015 inch to about 0.008 inch, or about 0.002 inch to about 0.006 inch.

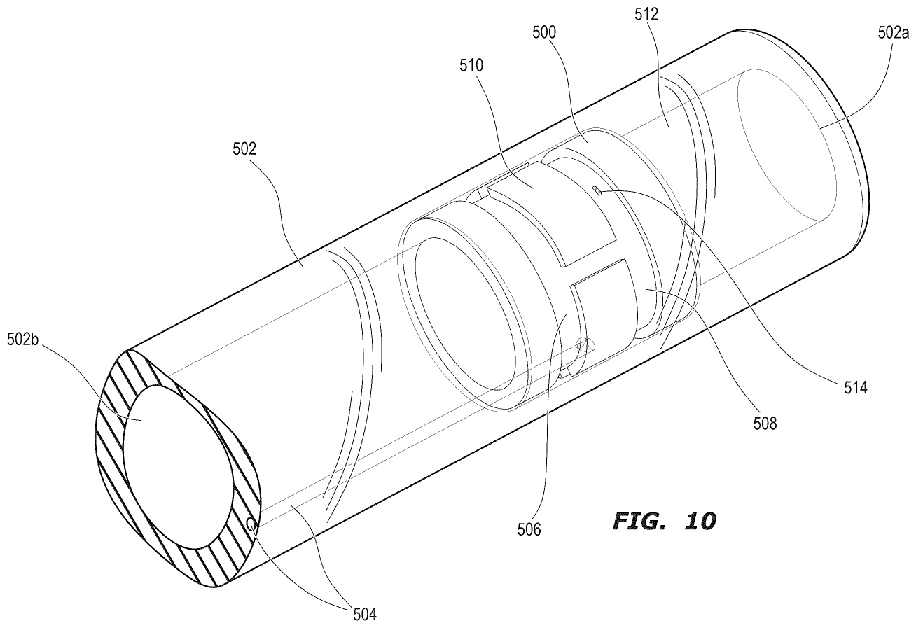

Referring now to FIGS. 10-12 another example embodiment of a jet rotator 500 is illustrated. FIG. 10 illustrates a view of the jet rotator 500 similar to the view of FIG. 9, while FIG. 11 illustrates a cross-sectional view taken along lines A-A from the top plan view of FIG. 12.

Similar to FIG. 9, FIG. 10 illustrates a jet rotator 500 that can be incorporated into system 100 or 200, or aspiration catheter 302. The jet rotator 500 is located radially towards a center of elongate shaft 502 (e.g., an aspiration catheter). In an embodiment, the jet rotator 500 is located towards a distal end 502a of the elongate shaft 502 and the supply lumen 504 runs along the proximal end 502b of the elongate shaft 502. The supply lumen 504 and the aspiration lumen 512 extend along the elongate shaft 502. The supply lumen 504 is connected to a pressurized fluid source (e.g., saline bag 224 of FIG. 4) which provides pressurized fluid to the supply lumen 504. Once the thrombotic material or thrombus has been broken apart, it exits through the aspiration lumen 512 by a vacuum connected to a collection canister (e.g., canister 218 of FIGS. 4-5).

The illustrated jet rotator 500 includes a proximal plenum 506 and a distal plenum 508 connected by one or more fluid channels 510. In the illustrated embodiment, the fluid channels 510 are parallel to one another, parallel to a longitudinal axis of the jet rotator 500, and/or generally perpendicular to plenums 506, 508, as compared to the helical channels 410 shown in FIG. 9. Like jet rotator 400, the jet rotator 500 includes a jet orifice 514, although het orifice 514 can be differently configured. As shown in FIGS. 11 and 12, the jet orifice 514 is formed so that the borehole forming orifice 514 is offset relative to the centerline of the jet rotator 500, so that the pressurized jet spray is directed or “aimed” away from the centerline of the aspiration lumen 512. This offset relative to the centerline of the aspiration lumen 512 applies a rotational force on the jet rotator 500 as the pressurized fluid flows through the jet orifice 514, causing the jet rotator 500 to rotate. Such rotation provides better coverage across the full cross-sectional area of the aspiration lumen, as compared to a rotationally static high pressure jet. As described with the earlier illustrated embodiments, the pressurized fluid is configured to break apart the thrombotic material or thrombus. In some embodiments, more than one jet orifice 514 may be provided, e.g., around the perimeter of the distal plenum 508 of jet rotator 500. In some embodiments, a jet orifice can be configured to be “aimed” at the centerline of the aspiration lumen, and another jet orifice can be configured to be “aimed” offset relative to the centerline, creating a rotational force applied to the jet rotator.

While principally described in context of delivering a jet of saline, it will be appreciated that other pressurized fluids (e.g., contrast media, or other) could also or alternatively be delivered using the described systems and methods, and such is within the scope of the present disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Following are some further example embodiments of the invention. These are presented only by way of example and are not intended to limit the scope of the invention in any way. Further, any example embodiment can be combined with one or more of the example embodiments.

Embodiment 1. A system for aspirating thrombus, comprising: an elongate shaft configured for placement within a blood vessel of a subject; a supply lumen and an aspiration lumen each extending along the shaft; a pressurized fluid source in fluid communication with the supply lumen to provide pressurized fluid to the supply lumen; and a jet rotator, wherein the jet rotator rotates when the pressurized fluid is allowed to flow through the supply lumen, the jet rotator comprising: a proximal plenum, wherein the proximal plenum is configured to allow injection of the pressurized fluid into the jet rotator when the pressurized fluid is caused or allowed to flow through the supply lumen; a distal plenum; one or more pressurized fluid channels, wherein the channels allow the pressurized fluid to flow from the proximal plenum to the distal plenum; and a jet orifice, wherein the jet orifice is configured to allow the pressurized fluid to flow from the distal plenum into the aspiration lumen.

Embodiment 2. The system of embodiment 1, wherein the jet rotator is located radially towards a center of the elongate shaft.

Embodiment 3. The system of any of embodiments 1-2, wherein the jet orifice has a cross-sectional dimension from about 0.002 inches to about 0.006 inches.

Embodiment 4. The system of any of embodiments 1-3, wherein the elongate shaft has an inner diameter of about 0.080 inches.

Embodiment 5. The system of any of embodiments 1-4, wherein the supply lumen provides the pressurized fluid at a pressure of about 3 MPa to about 6 MPa.

Embodiment 6. The system of any of embodiments 1-5, wherein the one or more channels have a helical shape.

Embodiment 7. The system of any of embodiments 1-6, wherein the pressurized fluid flowing through the one or more channels applies a rotational force on the jet rotator causing the jet rotator to rotate.

Embodiment 8. The system of any of embodiments 1-7, wherein the jet orifice is about 0.002 inches by about 0.006 inches.

Embodiment 9. The system of any of embodiments 1-8, wherein the jet rotator is located within the elongate shaft.

Embodiment 10. The system of any of embodiments 1-9, wherein a gap exists between the jet rotator and the elongate shaft, wherein some pressurized fluid leaks between the jet rotator and the elongate shaft to form a hydrodynamic bearing therebetween.

Embodiment 11. The system of embodiment 10, wherein the hydrodynamic bearing is configured to allow free rotation of the jet rotator.

Embodiment 12. The system of any of embodiments 1-11, wherein the jet orifice directs a pressurized jet of the pressurized fluid into the aspiration lumen, substantially centered on a centerline of the jet rotator.

Embodiment 13. The system of any of embodiments 1-12, wherein the jet orifice is offset relative to a centerline of the aspiration lumen.

Embodiment 14. The system of any of embodiments 1-13, wherein the pressurized fluid flowing through the jet orifice applies a rotational force to the jet rotator causing the jet rotator to rotate.

Embodiment 15. A method for aspirating thrombus, comprising: providing a system for aspirating a thrombus, the system comprising: an elongate shaft configured for placement within a blood vessel of a subject; a supply lumen and an aspiration lumen each extending along the shaft; a pressurized fluid source in fluid communication with the supply lumen to provide pressurized fluid to the supply lumen; and a jet rotator, wherein the jet rotator rotates when the pressurized fluid is allowed to flow through the supply lumen, the jet rotator comprising: a proximal plenum, wherein the proximal plenum is configured to allow injection of the pressurized fluid into the jet rotator when the pressurized fluid is caused or allowed to flow through the supply lumen; a distal plenum; one or more pressurized fluid channels, wherein the channels allow the pressurized fluid to flow from the proximal plenum to the distal plenum; and a jet orifice, wherein the jet orifice is configured to allow the pressurized fluid to flow from the distal plenum into the aspiration lumen; flowing the pressurized fluid to the supply lumen from the pressurized fluid source, wherein the pressurized fluid causes the jet rotator to rotate; macerating a thrombus located in the elongate lumen when the pressurized fluid contacts the thrombus; and aspirating the macerated thrombus.

Embodiment 16. The method of embodiment 15, wherein the jet rotator is located radially towards a center of the elongate shaft.

Embodiment 17. The method of any of embodiments 15-16, wherein the jet orifice has a cross-sectional dimension from about 0.002 inches to about 0.006 inches.

Embodiment 18. The method of any of embodiments 15-17, wherein the elongate shaft has an inner diameter of about 0.080 inches.

Embodiment 19. The method of any of embodiments 15-18, wherein the supply lumen provides the pressurized fluid at a pressure of about 3 MPa to about 6 MPa.

Embodiment 20. The method of any of embodiments 15-19, wherein the one or more pressurized fluid channels have a helical shape.

Embodiment 21. The method of any of embodiments 15-20, wherein the pressurized fluid flowing through the pressurized fluid channels applies a rotational force on the jet rotator causing the jet rotator to rotate relative to the aspiration lumen.

Embodiment 22. The method of any of embodiments 15-21, wherein the jet orifice is about 0.002 inches by about 0.006 inches.

Embodiment 23. The method of any of embodiments 15-22, wherein the jet rotator is located within the elongate shaft.

Embodiment 24. The method of any of embodiments 15-23, wherein a gap exists between the jet rotator and the elongate shaft, wherein some pressurized fluid leaks between the jet rotator and the elongate shaft to form a hydrodynamic bearing therebetween.

Embodiment 25. The method of embodiment 24, wherein the hydrodynamic bearing is configured to allow free rotation of the jet rotator.

Embodiment 26. The method of any of embodiments 15-25, wherein the jet orifice directs a pressurized jet of the pressurized fluid into the aspiration lumen, substantially centered on a centerline of the aspiration lumen.

Embodiment 27. The method of any of embodiments 15-26, wherein the jet orifice is offset relative to a centerline of the aspiration lumen.

Embodiment 28. The method of any of embodiments 15-27, wherein the pressurized fluid flowing through the jet orifice applies a rotational force to the jet rotator causing the jet rotator to rotate.

Embodiment 29. A jet rotator, wherein the jet rotator rotates when a pressurized fluid is allowed to flow through a supply lumen, the jet rotator comprising: a proximal plenum, wherein the proximal plenum is configured to allow injection of a pressurized fluid into the jet rotator when the pressurized fluid is caused or allowed to flow through a supply lumen; a distal plenum; one or more pressurized fluid channels, wherein the one or more pressurized fluid channels allow the pressurized fluid to flow from the proximal plenum to the distal plenum; and a jet orifice, wherein the jet orifice is configured to allow the pressurized fluid to flow from the distal plenum into an aspiration lumen.

Embodiment 30. The jet rotator of embodiment 29, wherein the jet orifice has a cross-sectional dimension from about 0.002 inches to about 0.006 inches.

Embodiment 31. The jet rotator of any of embodiments 29-30, wherein the one or more pressurized fluid channels have a helical shape.

Embodiment 32. The jet rotator of any of embodiments 29-31, wherein the jet orifice is about 0.002 inches by about 0.006 inches.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for aspirating thrombus, comprising:
an elongate shaft configured for placement within a blood vessel of a subject;
a supply lumen and an aspiration lumen each extending along the elongate shaft;
a pressurized fluid source in fluid communication with the supply lumen to provide pressurized fluid to the supply lumen; and
a jet rotator, wherein the jet rotator rotates when the pressurized fluid is allowed to flow through the supply lumen, the jet rotator comprising:
a proximal plenum, wherein the proximal plenum is configured to allow injection of the pressurized fluid into the jet rotator when the pressurized fluid is caused or allowed to flow through the supply lumen;
a distal plenum;
one or more pressurized fluid channels, wherein the channels allow the pressurized fluid to flow from the proximal plenum to the distal plenum; and
a jet orifice, wherein the jet orifice is configured to allow the pressurized fluid to flow from the distal plenum into the aspiration lumen.

2. The system of claim 1, wherein the jet rotator is located radially towards a center of the elongate shaft.

3. The system of claim 1, wherein the jet orifice has a cross-sectional dimension from about 0.002 inches to about 0.006 inches.

4. The system of claim 1, wherein the elongate shaft has an inner diameter of about 0.080 inches.

5. The system of claim 1, wherein the supply lumen provides the pressurized fluid at a pressure of about 3 MPa to about 6 MPa.

6. The system of claim 1, wherein the one or more channels have a helical shape.

7. The system of claim 1, wherein the pressurized fluid flowing through the one or more channels applies a rotational force on the jet rotator causing the jet rotator to rotate.

8. The system of claim 1, wherein the jet orifice is about 0.002 inches by about 0.006 inches.

9. The system of claim 1, wherein the jet rotator is located within the elongate shaft.

10. The system of claim 9, wherein a gap exists between the jet rotator and the elongate shaft, wherein some pressurized fluid leaks between the jet rotator and the elongate shaft to provide a hydrodynamic bearing therebetween.

11. The system of claim 10, wherein the hydrodynamic bearing is configured to allow free rotation of the jet rotator.

12. The system of claim 1, wherein the jet orifice directs a pressurized jet of the pressured fluid into the aspiration lumen, substantially centered on a centerline of the aspiration lumen.

13. The system of claim 1, wherein the jet orifice is offset relative to a centerline of the aspiration lumen.

14. The system of claim 13, wherein the pressurized fluid flowing through the jet orifice applies a rotational force to the jet rotator causing the jet rotator to rotate.

15. A method for aspirating thrombus, comprising:
providing a system for aspirating a thrombus, the system comprising:
an elongate shaft configured for placement within a blood vessel of a subject;
a supply lumen and an aspiration lumen each extending along the elongate shaft;
a pressurized fluid source in fluid communication with the supply lumen to provide pressurized fluid to the supply lumen; and
a jet rotator, wherein the jet rotator rotates when the pressurized fluid is allowed to flow through the supply lumen, the jet rotator comprising:
a proximal plenum, wherein the proximal plenum is configured to allow injection of the pressurized fluid into the jet rotator when the pressurized fluid is caused or allowed to flow through the supply lumen;
a distal plenum;
one or more pressurized fluid channels, wherein the channels allow the pressurized fluid to flow from the proximal plenum to the distal plenum; and
a jet orifice, wherein the jet orifice is configured to allow the pressurized fluid to flow from the distal plenum into the aspiration lumen;
flowing the pressurized fluid to the supply lumen from the pressurized fluid source, wherein the pressurized fluid causes the jet rotator to rotate;
macerating a thrombus located in the aspiration lumen when the pressurized fluid contacts the thrombus; and
aspirating the macerated thrombus.

16-20. (canceled)

21. The method of claim 20, wherein the pressurized fluid flowing through the pressurized fluid channels applies a rotational force to the jet rotator causing the jet rotator to rotate relative to the aspiration lumen.

22-28. (canceled)

29. A jet rotator, wherein the jet rotator rotates when a pressurized fluid is allowed to flow through a supply lumen, the jet rotator comprising:
a proximal plenum, wherein the proximal plenum is configured to allow injection of the pressurized fluid into the jet rotator when the pressurized fluid is caused or allowed to flow through the supply lumen;
a distal plenum;
one or more pressurized fluid channels, wherein the one or more pressurized fluid channels allow the pressurized fluid to flow from the proximal plenum to the distal plenum; and
a jet orifice, wherein the jet orifice is configured to allow the pressurized fluid to flow from the distal plenum into an aspiration lumen.

30. The jet rotator of claim 29, wherein the jet orifice has a cross-sectional dimension from about 0.002 inches to about 0.006 inches.

31. The jet rotator of claim 29, wherein the one or more pressurized fluid channels have a helical shape.

32. The jet rotator of claim 29, wherein the jet orifice is about 0.002 inches by about 0.006 inches.
